(19) United States
(12) Patent Application Publication
(10) Pub. No.: US 2009/0306219 A1
(43) Pub. Date: Dec. 10, 2009

(54) TOPICAL USE OF PLANT EXTRACT FOR SKIN DETOXIFICATION

(76) Inventor: Richard Presti, Airmont, NY (US)

Correspondence Address: LADAS & PARRY LLP, 26 WEST 61ST STREET, NEW YORK, NY, 10023, US

(21) Appl. No.: 12/132,895

(22) Filed: Jun. 4, 2008

(51) Int. Cl. A61K 31/10; A61P 17/18

(52) U.S. Cl. 514/706

(57) ABSTRACT

Specific plant extracts are applied topically to upregulate genes which code for proteins to prevent the build-up of and mitigate the activity of various harmful materials within the skin. These upregulated proteins include glutathione transferases, peroxiredoxins, oxidoreductases, glutaredoxins and glutathione syntheses. These proteins produce additional glutathione, glyoxalase I, maintain cellular redox homeostasis, reduce hydrogen peroxide and alkylhydroperoxides, and conjugate glutathione to a wide number of exogenous and endogenous hydrophobic electrophiles. Keratinocytes have been induced to significantly upregulate glutathione and glyoxalase I protein expression. Additionally, keratinocytes pre-treated with diallyl trisulfide have increased viability when exposed to ozone, UV radiation or cigarette smoke extract. The active material can be extracted from plants such as garlic, onions, shallots, leeks, chives, scallions, brussel sprouts, broccoli, cabbage, cauliflower, bok choy, kale, mustard, turnip or radish, may include any compound of the allyl sulfide family, and may be of synthetic origin.

### FIELD OF THE INVENTION

The present invention relates to the topical application of plant extract and, more particularly, to use allyl sulfides topically to upregulate cellular detoxification genes. Allyl sulfides can be synthesized or more preferably isolated from a number of plant sources including but not limited to garlic, onions, shallots, leeks, chives, scallions, brussel sprouts, broccoli, cabbage, cauliflower, bok choy, kale, mustard, turnip and radish.

### BACKGROUND OF THE INVENTION

The skin is the largest organ of the human body. Its myriad functions include protection from the environment and pathogens, sensations of hot, cold and touch, heat regulation by radiation or constriction of blood vessels, prevention of evaporation, synthesis of vitamins B and D, storage of lipids and water and excretion of waste materials. Being the first line of defense, the skin is continually bombarded by extrinsic factors including UV irradiation, smog, pollution, cigarette smoke, and ozone, pathogens and xenobiotic substances and harsh cleansers and changes in humidity. Additionally, these exogenous insults are compounded by internal buildups of cellular respiratory by-products including reactive oxygen species. These insults in total effect the functionality and appearance of the skin. Over time there is a reduction of collagen, elastin and glycosaminoglycans, the dermal and epidermal layers of the skin thin, and the barrier function is reduced. This results in a slackening and wrinkling of the skin and furthers the inability of the skin to deal with these damaging insults.

A plethora of cosmetic products are available to the consumer that offer skin benefits such as improved hydration, UV protection, anti-aging materials and peptides and ingredients to mitigate wrinkles. Additionally, plastic surgeons are able to perform a variety of procedures from microderm abrasions to full face lifts.

These other solutions have their draw backs. Cosmetic claims are often overstated and often only offer protection from one environmental insult. More specifically, cosmetic ingredients may work well in the laboratory, however, the actives are more often than not unable to penetrate the skin to reach the site where they are needed. Furthermore, due to the vast environmental and endogenous insults imparted on the skin, it is highly unlikely that a single or select few concoction of raw materials will have the flexibility to neutralize the myriad detrimental agents skin cells are exposed to, however over millions of years evolution has devised mechanisms to allow cells to survive harsh, oxidative environmental conditions. Finally, many cosmetic products address physiological damages only after they occur, but do nothing to prevent them from happening.

It is therefore an object of this invention to provide a cosmetic material to aid in the prevention of formation and/or mitigation of both intrinsic and extrinsic molecules and materials that can have a detrimental effect on the structure, longevity or overall function of the skin. Advantages to this approach include:
The active material may be isolated from a natural source
There is no need to overcome the issue of penetration of an active molecule into the skin
Whereas commonly used cosmetic ingredients have specific activity against one or a few environmental or intrinsic agents or toxins, the upregulation of the cell's own detoxification system can eliminate virtually every toxin or xenobiotic substance the cell is exposed to.

The primary cell type of the epidermis is the keratinocyte, which constitutes approximately 90% of epidermal cells. These keratinocytes originate from the basal layer through differentiation of keratinocyte stem cells. Through differentiation, keratinocytes enucleate, flatten and become highly keratinized, forming the stratum corneum which forms a protective layer against endogenous insults. The skin's keratinocytes have innate defenses to protect against toxins and/or xenobiotic substances. Whereas it can be beneficial to apply cosmetically active materials to the skin to aid on any detoxification event, it would be most advantageous to enable or entice the keratinocytes to produce additional detoxification proteins.

### SUMMARY OF THE INVENTION

In accordance with the present invention, allyl sulfides can be extracted from plant sources including but not limited to garlic, onions, shallots, leeks, chives, scallions, brussel sprouts, broccoli, cabbage, cauliflower, bok choy, kale, mustard, turnip or radish or synthetically derived and can be applied topically to the skin to upregulate genes which code for proteins that function to prevent and mitigate various harmful materials within the skin. These upregulated genes can code for proteins which can include but are not limited to glutathione transferases, peroxiredoxins, oxidoreductases, glutaredoxins and glutathione syntheses. These proteins function independently of or in conjunction with one another to produce additional glutathione, maintain cellular redox homeostasis, reduce hydrogen peroxide and alkylhydroperoxides, and to conjugate glutathione to a wide number of exogenous and endogenous hydrophobic electrophiles.

### DESCRIPTION OF THE INVENTION

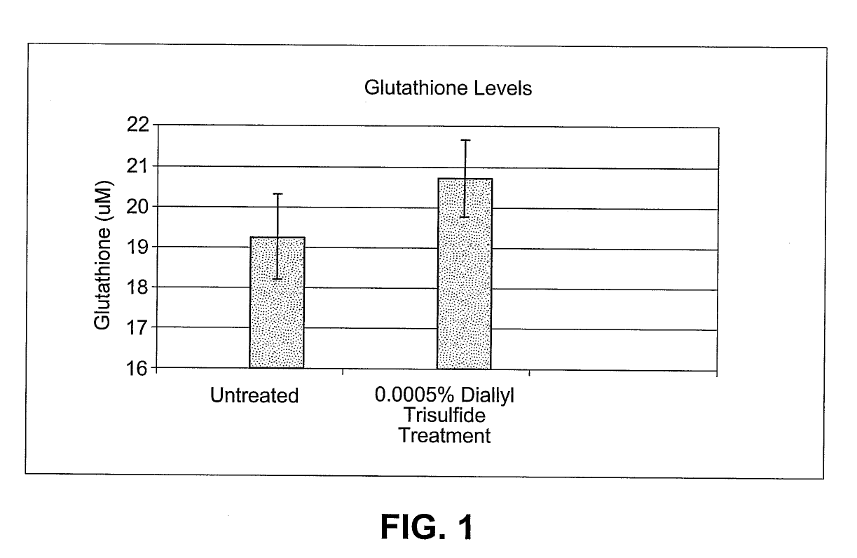

FIG. 1 is a graphic representation of Elisa quantification of glutathione.

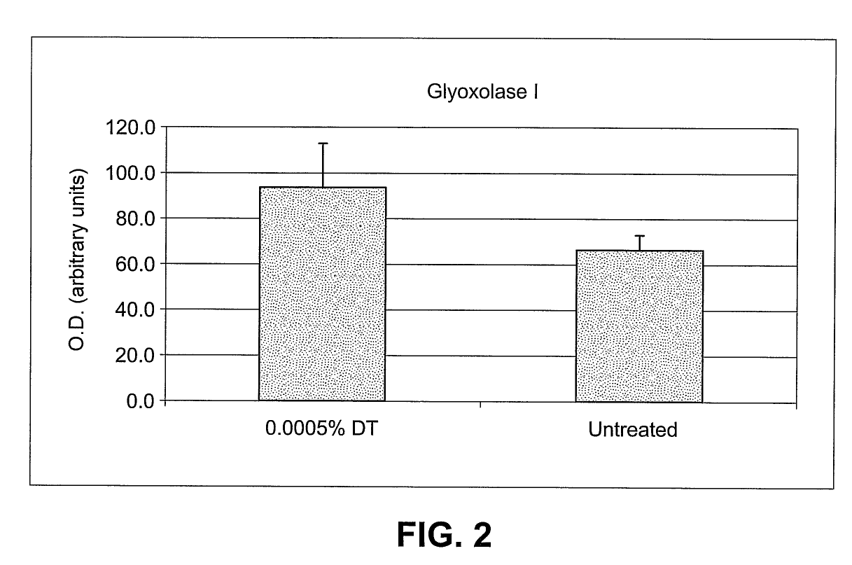

FIG. 2 is a graphic representation of quantification of Glyoxalase I vis immunoassay.

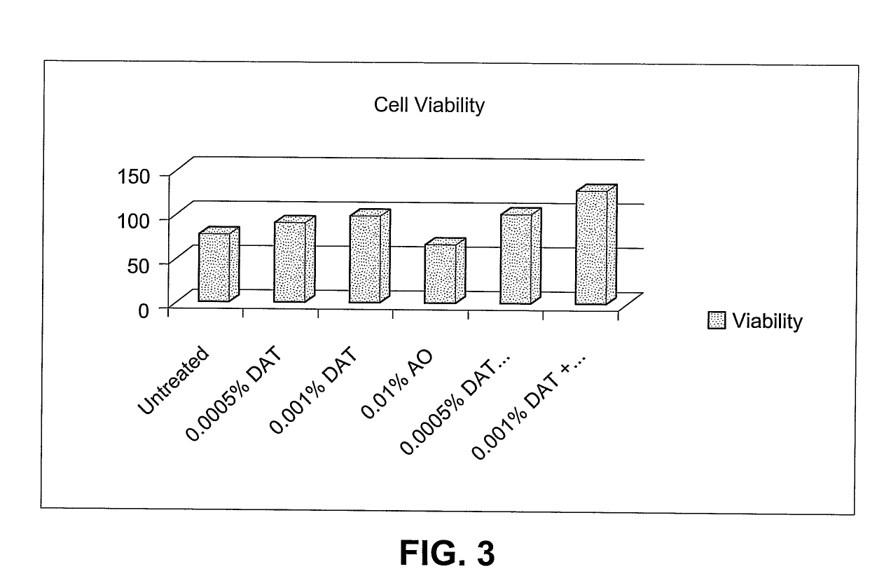

FIG. 3 is a graphic representation of keratinocyte cell culture viability after exposure to cigarette smoke extract, as determined via MTT assay.

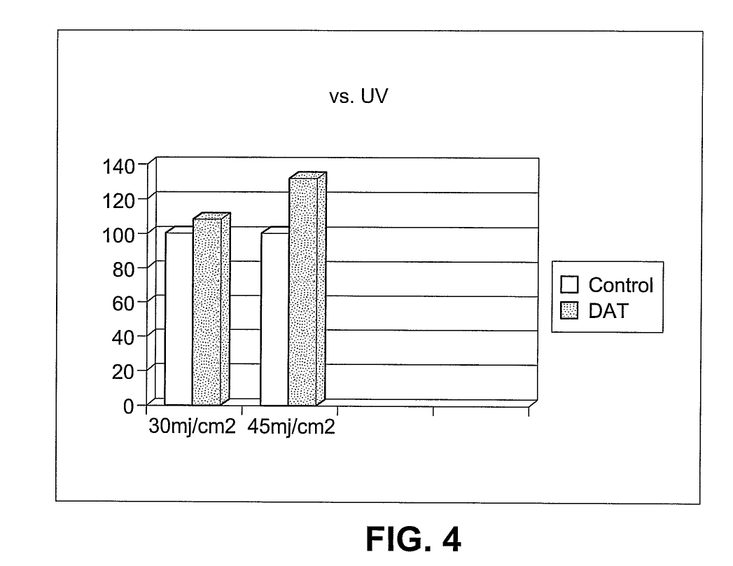

FIG. 4 is a graphic representation of keratinocyte cell culture viability after exposure to UV radiation, as determined via MTT assay.

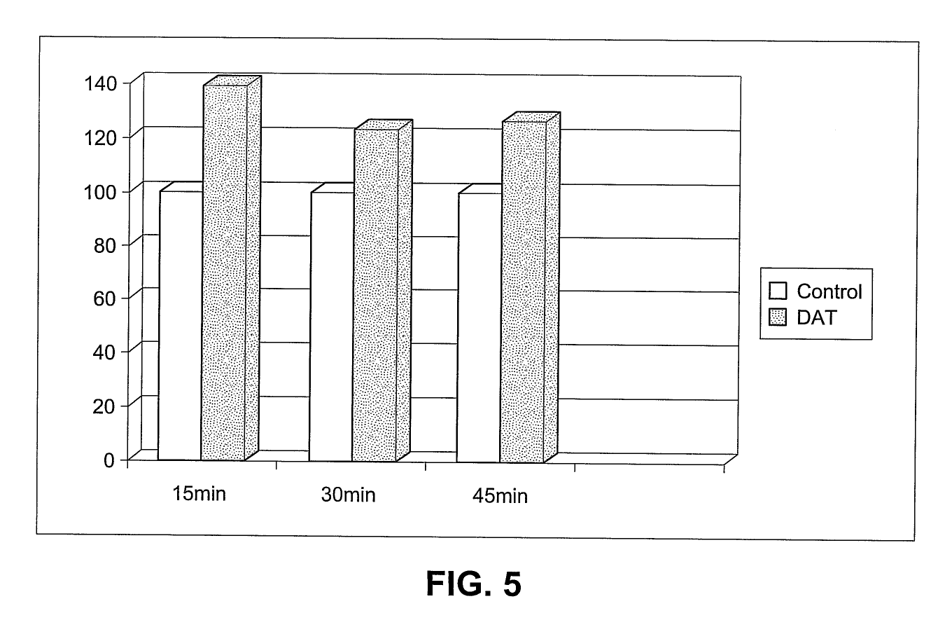

FIG. 5 is a graphic representation of keratinocyte cell culture viability after timed exposure to 10 ppm ozone, as determined via MTT assay.

### DETAILED DESCRIPTION OF INVENTION

### EXAMPLE 1

### Results of DNA Microarray

### Summary of Test Method

DNA microarrays are extremely powerful tools that allow users to analyze changes in gene expression by monitoring the RNA products of thousands of genes in a single experiment. Microarrays come in many forms, however the most popular form are comprised of glass microscope slides which have been studded with a large number of DNA fragments, also called features, with each feature containing a nucleotide sequence that corresponds to a single specific gene. DNA microarrays are commonly used to compare treated and untreated cells/tissue to determine what changes in gene expression occur with the treatment.

The first step in the microarray process is to decide on which cell or tissue type to use as the model. For example, if one wishes to look at the effect of a material on keratinocyte gene expression then one can use a keratinocyte cell culture model (keratinocytes grown in a monolayer), or a keratinocyte culture. In the first two examples the microarray will show only the changes in keratinocyte genes.

After deciding upon a model, the next phase is the treatment phase. Typically, when a material stimulates a change in gene expression changes in the mRNA for that specific gene can be observed within 12-24 hours. Thus the treatment period for most microarray studies are very short.

After purifying the RNA, mRNA must be isolated from the total RNA, and then the mRNA must be converted into antisense RNA (aRNA) so that it can bind to the DNA fragments on the microarray.

After the aRNA is generated it is labeled with a fluorescent probe. Once the aRNA is labeled and any unincorporated dye is removed from the sample, the labeled aRNA probe is mixed with a hybridization solution and applied to the microarray. The microarray is then hybridized overnight at a high temperature (normally 60-65° C.). The combination of the hybridization solution (high salt solution) and the high temperature promotes conditions of very high stringency. This allows only those aRNA and DNA fragments to interact and bind via Watson-Crick base pairs that share exact complimentary base sequences. After hybridization the microarray is washed to remove any unbound aRNA probe and then scanned with a microarray scanner.

The table below lists Examples of genes upregulated with plant extract (cDNA microarray results)

Keratinocyte

| Gene | Upregulation |
|---|---|
| Glutathione s-transferase a4 | 1.697 |
| Defensin, beta 4 | 2.227 |
| Defensin, beta 129 | 1.868 |
| Peroxiredoxin | 1.684 |
| Putative Oxidoreductase | 1.761 |
| Glutathione s-transferase theta 1 | 1.516 |
| cytokine induced apoptosis inhibitor 1 | 1.438 |
| p53 associated parkin-like cyto. Protein | 1.337 |
| glutaredoxin 2 | 1.542 |
| Heat shock transcription factor 1 | 1.653 |
| defender against cell death 1 | 1.97 |
| tumor protein p53 inducible nuclear | 1.872 |
| protein 1 | |
| cold inducible rna binding protein | 1.407 |
| nucleophosmin/nucleoplasmin 3 | 1.377 |
| hsp 40 | 1.405 |
| heat shock protein 90 kda alpha | 1.607 |
| (cytostolic) | |
| Glutathione synthetase | 1.437 |

### EXAMPLE 2

### ELISA Quantification of Glutathione

In simple terms, in ELISA an unknown amount of antigen is affixed to a surface, and then a specific antibody is washed over the surface so that it can bind to the antigen. In this case, an ELISA was performed to quantify the amount of glutathione expressed in keratinocyte culture in a controlled culture as well as after 24 hrs treatment with 0.0005% dially trisulfide. This antibody is linked to an enzyme, and in the final step a substance is added that the enzyme can convert to some detectable signal. Thus in the case of fluorescence ELISA, when light is shone upon the sample, any antigen/antibody complexes will fluoresce so that the amount of antigen in the sample can be measured.

The ELISA kit used in this experiment was purchased from Cayman Chemicals. The experiment was performed as per the manufacturer's directions. The results are shown in the following table and graphed in FIG. 1:

| 48 hours | | | | | | | | |
|---|---|---|---|---|---|---|---|---|
| Treatment | | | | Glut | Glut | Glut | | |
| Treatment | abs 1 | abs 2 | abs 3 | 1 | 2 | 3 | Mean | Stdev |
| Untreated | 0.030 | 0.033 | 0.033 | 18 | 20 | 20 | 19 | 1 |
| 0.0005% | 0.033 | 0.036 | 0.034 | 20 | 22 | 20 | 21 | 1 |
| Diallyl | | | | | | | | |
| Trisulfide | | | | | | | | |

### EXAMPLE 3

### Quantification of Glyoxalase I Via Immunoassay

To quantify the amount of glyoxalase I which was upregulated vs. a control sample, a keratinocyte culture was treated for 24 hrs, lysed, and western blotted vs. an untreated control sample. The blots were then transferred and probed with a fluorescent anti-body to glyoxalse I, purchased from Santa Cruz Biotechnology. Absorbance was measured and graphed. Results are shown in the following tables and graphed in FIG. 2:

| Treatment | OD1 | OD 2 | OD 3 | OD4 | OD 5 | OD 6 |
|---|---|---|---|---|---|---|
| 0.0005% | 125.205 | 126.011 | 145.641 | 87.372 | 123.78 | 130.525 |
| DT | | | | | | |
| Untreated | 153.701 | 152.977 | 158.406 | 149.661 | 147.67 | 139.384 |

| Inv | Inv | Inv | Inv | Inv | Inv | | | |
|---|---|---|---|---|---|---|---|---|
| OD 1 | OD 2 | OD 3 | OD 4 | OD 5 | OD 6 | Mean | Stdev | |
| 91.6 | 90.7 | 71.1 | 129.4 | 93.0 | 86.2 | 93.7 | 19.3 | * significantly |
| | | | | | | | | different from |
| | | | | | | | | Untreated |
| 63.1 | 63.8 | 58.4 | 67.1 | 69.1 | 77.4 | 66.5 | 6.5 | |

### EXAMPLE 4

### Keratinocyte Viability in the Presence of 15% Cigarette Smoke Extract

Cigarette smoke extract (CSE) was prepared by inhaling 2 Marlboro Light cigarettes through 30 mL Dulbecco's Modified Eagle's Medium (SIGMA). This CSE was then incorporated into keratinocyte cultures that had been grown to confluence and pre-treated for 24 hrs with either:
1. Untreated (control)
2. 0.0005% Diallyl trisulfide
3. 0.001% Diallyl trisulfide
4. 0.01% commercially available anti-oxidant
5. 0.0005% Diallyl trisulfide and 0.01% commercially available anti-oxidant
6. 0.001% Diallyl trisulfide and 0.01% commercially available anti-oxidant

Cell viability was determined via an MTT assay, and the results graphed in FIG. 3.

### EXAMPLE 5

### Keratinocyte Viability in the Presence of UV Irradiation

Keratinocytes were grown to confluence, and treated with:
1. No treatment (control)
2. 0.0005% Diallyl trisulfide for 24 hrs

These cell cultures were exposed to UV radiation at either:
1. 30mj/cm2
2. 45mj/cm2

Keratinocyte viability was determined via an MTT assay, and the results graphed in FIG. 4

### EXAMPLE 6

### Keratinocyte Viability in the Presence of 10 ppm Ozone

Keratinocytes were grown to confluence, and treated with:
1 No treatment (control)
2 0.0005% Diallyl trisulfide for 24 hrs

These cell cultures were exposed to 10 ppm Ozone for either:
1. 15 min
2. 30 min
3. 45 min

Keratinocyte viability was determined via an MTT assay, and the results graphed in FIG. 5.

The invention is not considered limited to the examples chosen for purposes of disclosure and illustrations, and covers all changes and modifications which do not constitute departures from the true spirit and scope of this invention.

Having thus described the invention, what is desired to be protected by Letters Patent is presented in the subsequently appended claims.

1-7. (canceled)

8. A topical formulation comprising an upregulation agent for at least one gene that codes for proteins that function to prevent buildup and mitigate the activity of harmful materials to the skin, the upregulation agent being an allyl sulfide.

9. The topical formulation of claim 8 wherein said allyl sulfide is isolated or extracted from a plant source

10. The topical formulation of claim 9 wherein said allyl sulfide is isolated from a plant selected from the group consisting of garlic, onions, shallots, leeks, chives, scallions, brussel sprouts, broccoli, cabbage, cauliflower, bok choy, kale, mustard, turnip, and radish.

11. The topical formulation of claim 8 wherein the allyl sulfide is synthesized.

12. The topical formulation of claim 8 wherein said allyl sulfide comprises diallyl trisulfide.

13. The topical formulation of claim 8 wherein the upregulation agent upregulates the expression of glutathione.

14. The topical formulation of claim 8 wherein said upregulation agent affects at least one gene that codes for a protein which can upregulate the expression of glyoxalase 1.
